(19) United States
(12) Patent Application Publication
(10) Pub. No.: US 2010/0204764 A1
(43) Pub. Date: Aug. 12, 2010

(54) Method for Treating Hot Flashes Associated with Menopause During Sleep

(76) Inventor: Bruce A. Garetz, New York, NY (US)

Correspondence Address: SMITH, GAMBRELL & RUSSELL, SUITE 3100, PROMENADE II, 1230 PEACHTREE STREET, N.E., ATLANTA, GA, 30309-3592, US

(21) Appl. No.: 12/697,663

(22) Filed: Feb. 1, 2010

Related U.S. Application Data
(60) Provisional application No. 61/151,565, filed on Feb. 11, 2009.

(51) Int. Cl. A61F 7/00

(52) U.S. Cl. 607/104

(57) ABSTRACT

A method and device for automatically detecting the onset of a hot flash while the subject is sleeping, and of cooling the subject using a cold fluid circulating through a vest or a mattress pad, before sweating and/or chills develop and awaken the subject. A temperature sensor is attached to the subject's skin, and is connected to a computer or microprocessor. The subject dons a vest that is connected to a coolant circulation system. Alternatively, the subject lies on a mattress pad that is connected to a coolant circulation system. During the night, if the subject's skin temperature rises more than a predetermined amount within a time period, or rises above a predetermined threshold temperature, the coolant circulation system is activated for a fixed period of time. The cooled vest or pad helps to draw heat away from the subject's skin and thus reduce the symptoms of the hot flash. After the circulation terminates, the system is reset to monitor the skin temperature once again. The cycle can be repeated any number of times during the night. Instead of a temperature sensor, a humidity sensor can also be used.

### CROSS REFERENCE TO RELATED PATENT APPLICATIONS

This patent application claims priority from U.S. Provisional Patent Application No. 61/151,565, filed Feb. 11, 2009, which is hereby incorporated by reference.

### BACKGROUND OF THE INVENTION

1. Technical Field

This invention relates generally to methods and devices for treating the effects of menopause and relates more specifically to methods and devices for automatically detecting the onset of a hot flash while the subject is sleeping and of cooling the subject before sweating and/or chills develop and awaken the subject.

Three out of four of post-menopausal women suffer from hot flashes. Hot flashes are caused by problems in the temperature regulating system of the body due to decreased hormone levels. Hot flashes result in short episodes of blood vessel dilation and temperature rise in the skin, followed by sweating and chills. These symptoms can cause loss of sleep. The invention pertains to a method of automatically detecting the onset of a hot flash while the subject is sleeping, and of cooling the subject using a cold fluid circulating through a vest or a mattress pad, before the sweating and chills develop and awaken the subject.

2. Prior Art

Almost all past approaches to treating hot flashes associated with menopause have involved pharmaceutical therapies, such as hormone replacement therapy and alternative botanical therapy. While hormone replacement therapy is effective, it can have serious side effects such as increasing the risk of breast cancer, heart disease, and stroke. Botanical and other chemical therapies are of questionable efficacy and can have their own adverse side effects.

A few patents report wrist bands filled with coolant or cooling patches that are attached to the back between the shoulder blades. Because both the wrist bands and patches require the subject to sense the onset of a hot flash and to then attach the band or patch, they are of no use during sleep. Also, both these bands and patches contain sealed compartments filled with coolants that are activated by the subject, and can only be used once.

U.S. Pat. No. 5,956,963 to Lerner discloses a wrist cooler for relief of hot flashes and similar symptoms. The invention offers relief from hot flash symptoms of menopause and body overheating by providing a wrist cooler that preferably includes chemical cooling pellets that remain in a solid state until broken. A woman experiencing symptoms such as menopausal hot flashes carries the cooler in her purse and, when hot flashes occur, she removes the cooler from her purse, breaks the pellets, and slides the cooler onto her wrist. Once the hot flashes have subsided, the cooler is discarded, because there are no reusable cooling elements to be frozen or chilled.

U.S. Patent Publication No. 2007/0098769 to Champion discloses systems and methods for treating hot flashes associated with menopause. The invention comprises kits and systems for treating hot flashes in a menopausal woman including one or more cooling devices suitable to be placed on a woman's skin, at a hot flash origin site, when she experiences a hot flash or anticipates a hot flash. The cooling device or devices may be provided in a sealed package and may be in the form of an adhesive patch that can adhere to the woman's skin.

Although unrelated to the treatment of menopause, U.S. Pat. No. 4,691,762 to Elkins et al. discloses a personal temperature control system including a first heat exchanger adapted to be worn as a garment and a second heat exchanger adapted to interact with a temperature source, the first and second heat exchangers being interconnected through a pump and reservoir unit. The heat exchangers together with the pump and reservoir are portable.

An article by R. R. Freedman and S. Wasson entitled “Miniature hygrometric hot flash recorder”, Fertil. Steril. 2007 88(2) 494-496, discloses a miniature ambulatory hot flash recorder that uses neither electrodes nor gel that will operate for thirty-one days on a single hearing aid battery. In the first study, putative hot flashes recorded using a relative humidity sensor were compared with patient event marks. In the second study, relative humidity recorded by using a complete prototype recorder was compared with sternal skin conductance recordings made on a Biolog recorder, as well as with event marks. Ten healthy postmenopausal women reporting frequent hot flashes and using no medication were studied. The authors claim that their method's accuracy is comparable to that achieved by skin conductance level (SCL) measurements but that their method is simpler and much easier for patients to utilize.

Accordingly, there is a need for improved methods and devices for treating hot flashes associated with menopause during sleep. It is to these needs, among others, that this invention is directed.

### BRIEF SUMMARY OF THE INVENTION

The invention pertains to a method of automatically detecting the onset of a hot flash while the subject is sleeping, and of cooling the subject using a cold fluid circulating through a vest or a mattress pad, before the sweating and chills develop and awaken the subject. A temperature sensor is attached to the subject's skin, and is connected to a computer or microprocessor. Before bedtime, the subject dons a vest that is connected to a coolant circulation system. Alternatively, the subject lies on a mattress pad that is connected to a coolant circulation system. During the night, the subject's skin temperature is monitored periodically. If the skin temperature rises more than a predetermined amount within the time period, or rises above a predetermined threshold temperature, the coolant circulation system is activated for a fixed period of time. The cooled vest or pad helps to draw heat away from the subject's skin and thus reduces the symptoms of the hot flash. After the circulation terminates, the system is reset to monitor the skin temperature once again. The cycle can be repeated any number of times during the night. Alternatively, instead of a temperature sensor, a humidity sensor can also be used.

Other features, aspects, and advantages of the invention will become apparent from the following detailed description of the preferred embodiments, taken in conjunction with the accompanying drawings.

### BRIEF DESCRIPTION OF THE FIGURES

FIG. 1 shows a first embodiment of this invention comprising a cooling vest.

FIG. 2 shows a second embodiment of this invention comprising a cooling pad.

### DETAILED DESCRIPTION OF PREFERRED EMBODIMENTS

Reference will now be made in detail to illustrative preferred embodiments of the invention, examples of which are illustrated in the accompanying drawings. While the invention will be described in conjunction with the preferred embodiments, it will be understood that they are not intended to limit the invention to those embodiments. On the contrary, the invention is intended to cover alternatives, modifications, and equivalents, which may be included within the spirit and scope of the invention.

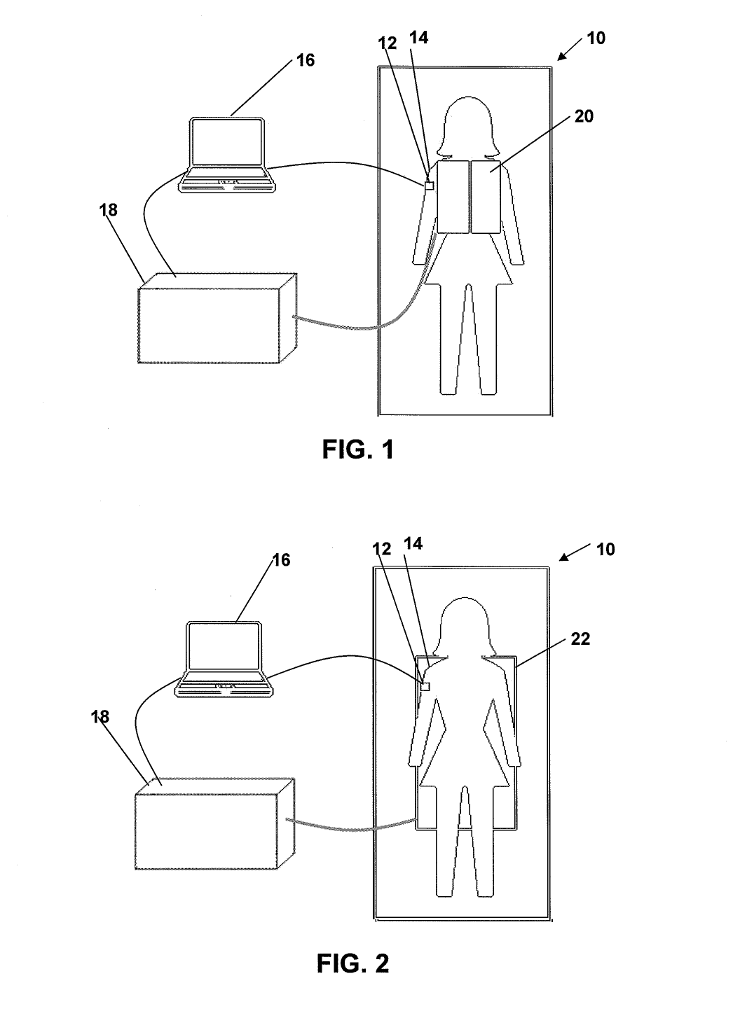

Two illustrative embodiments will be used to disclose the invention. A first embodiment is shown in FIG. 1, which comprises the use of a cooling vest. A second embodiment is shown in FIG. 2, which comprises the use of a cooling pad. Other cooling devices are contemplated and are included in the scope of the invention.

Referring now to the figures, illustrative embodiments of the invention 10 comprise:
1) a temperature and/or a humidity sensor 12 attached to the skin of the subject 14;
2) a microprocessor 16 that is programmed to periodically record the signal(s) from the sensor(s) 12 and to compare them with preset conditions to determine if a hot flash has commenced; and
3) a cooling unit 18 that circulates cool, temperature-controlled fluid through a heat exchanger comprising either a) a vest 20 that is worn by the subject 14 during sleep, or b) a mattress pad 22 on which the subject 14 sleeps.

The sensor 12 (the invention 10 can comprise either or both of the temperature and humidity types of sensors) is attached to the subject's 14 skin, and is connected to the computer or microprocessor 16 either by wire or wirelessly, typically through an analog-to-digital converter. Before bedtime, the subject 14 dons a vest 20 that is connected to the cooling unit 18. Alternatively, the subject 14 lies on a mattress pad 22 that is connected to the cooling unit 18. During the night, the subject's 14 skin temperature is monitored periodically. The time period can be on the order of seconds or minutes. If the skin temperature rises more than a predetermined amount within the time period, or rises above a predetermined threshold temperature, the cooling unit 18 is activated for a fixed period of time. The cooled vest 20 or pad 22 helps to draw heat away from the subject's 14 skin and thus reduce the symptoms of the hot flash. After the circulation terminates, the system is reset to monitor the skin temperature once again. The cycle can be repeated any number of times during the night.

The time period for which the cooling unit 18 is activated can be on the order of seconds or minutes. The condition for the temperature sensor 12 can be either a) that the subject's 14 skin temperature has exceeded a particular preset value or b) that the subject's 14 skin temperature change between the current and previous measurement has exceeded a particular preset value. The condition for the humidity sensor 12 can be either a) that the subject's 14 skin relative humidity (or the relative humidity at a point immediately proximal to the subject's 14 skin) has exceeded a particular preset value or b) that the subject's 14 skin relative humidity (or the relative humidity at a point immediately proximal to the subject's 14 skin) change between the current and previous measurement has exceeded a particular preset value. If both temperature and humidity sensors 12 are used, the condition for determining the onset of the hot flash can be either a) that both sensor 12 values have exceeded their preset values or b) that either sensor 12 value has exceeded its preset value.

If no onset of a hot flash is detected, the microprocessor 16 continues to periodically record sensor 12 signals. When the onset of a hot flash is detected, the microprocessor 16 sends a signal to commence a cooling cycle (lasting for 1 to 10 minutes) of the cooling unit 18. When the cooling cycle is complete, the microprocessor 16 pauses for a predetermined time period to allow the vest 20 or pad 22 to return to room temperature, and then returns to its mode of periodically recording sensor 12 signals and comparing them with preset conditions to determine if another hot flash has commenced.

The contact of the cool vest 20 or pad 22 with the subject's 14 skin relieves that discomfort associated with the hot flash, reduces the sweating and chills normally associated with the hot flash, and prevents the subject 14 from awakening. The invention 10 could also be used to automatically treat a subject for hot flashes while awake.

A prototype of this invention 10 has been built using a laptop computer running LabView and Vernier LoggerPro software to simulate the microprocessor 16 functions. A Vernier surface temperature sensor 12, consisting of an exposed thermister, was taped to the subject's 14 underarm, connected by wire to a Vernier LabPro interface, plugged into the laptop computer. The subject 14 was also wearing a Game Ready™ Active Cooling Vest 20 connected to a Game Ready™ Control Unit (cooling unit 18) that was filed with ice and water, and preset to run 5-minute cooling cycles.

The temperature of the subject's 14 skin was measured very few seconds. When the measured temperature exceeded 35 degrees Celsius, the laptop activated two electrical solenoid actuators in sequence, one second apart, which pressed two pressure-activated switches on the Game Ready Control Unit and commenced a 5-minute cooling cycle. The solenoids were activated using a Vernier Digital Control Unit (DCU), connected to the LabPro and a DC power supply. When a digital command was sent from the laptop, the DCU closed an analog switch, applying a DC voltage to the solenoids for one-second each, thus activating the solenoids.

After the cooling cycle was completed, a programmed 20-minute pause allowed the vest 20 to return to room temperature. After the 20-minute pause, the monitoring of the temperature of the sensor 12 was resumed, so that the system was ready to detect the next hot flash.

Given that 75% of all post-menopausal women could be helped by this biomedical invention, the potential market is enormous. Compared to drug therapies, the risks of the invention are negligible.

While the invention has been described in connection with certain preferred embodiments, it is not intended to limit the spirit or scope of the invention to the particular forms set forth, but is intended to cover such alternatives, modifications, and equivalents as may be included within the true spirit and scope of the invention.

What is claimed is:

1. A method for treating hot flashes associated with menopause during sleep comprising the steps of:
a. automatically detecting the onset of a hot flash while the subject is sleeping, and
b. cooling the subject using a cold fluid circulating through a heat exchanger before sweating and/or chills develop and awaken the subject.

2. The method as claimed in claim 1, wherein the heat exchanger is a vest.

3. The method as claimed in claim 1, wherein the heat exchanger is a mattress pad.

4. The method as claimed in claim 1, wherein detecting the onset of the hot flash includes monitoring skin temperature of the subject.

5. The method as claimed in claim 1, wherein detecting the onset of the hot flash includes monitoring of the relative humidity adjacent to the subject.

6. The method as claimed in claim 1, wherein detecting the onset of the hot flash includes monitoring skin temperature of the subject and relative humidity adjacent to the subject.

7. A device for treating hot flashes associated with menopause while a subject is sleeping comprising:
a. a sensor for monitoring the subject for the onset of a hot flash and generating a sensor signal;
b. a microprocessor that is programmed to periodically sample the sensor signal and to compare the sensor signal with preset conditions to determine if a hot flash has commenced; and
c. a cooling unit that circulates cool, temperature-controlled fluid through a heat exchanger adjacent to the subject.

8. The device as claimed in claim 7, wherein the heat exchanger is a vest.

9. The device as claimed in claim 7, wherein the heat exchanger is a mattress pad.

10. The device as claimed in claim 7, wherein the sensor is a temperature sensor.

11. The device as claimed in claim 7, wherein the sensor is a relative humidity sensor.

12. The device as claimed in claim 7, wherein the sensor includes both a temperature sensor and a relative humidity sensor.

13. The device as claimed in claim 7, wherein the sensor is either wired or wirelessly connected to the microprocessor through an analog-to-digital converter.
